(19) United States
(12) Patent Application Publication
(10) Pub. No.: US 2022/0370465 A1
(43) Pub. Date: Nov. 24, 2022

(54) TREATMENT OF MAST CELL DISEASES AND EOSINOPHILIC DISORDERS

(71) Applicant: Blueprint Medicines Corporation, Cambridge, MA (US)

(72) Inventors: Brenton Mar, Cambridge, MA (US); Alexandra Grassian, Cambridge, MA (US)

(21) Appl. No.: 17/773,447

(22) PCT Filed: Nov. 3, 2020
(86) PCT No.: PCT/US2020/058632

§ 371 Date: Apr. 29, 2022

Related U.S. Application Data
(60) Provisional application No. 62/930,338, filed on Nov. 4, 2019.

(51) Int. Cl. A61K 31/53

(52) U.S. Cl. CPC A61K 31/53

(57) ABSTRACT

The present disclosure relates to the use of Compound (I) or a pharmaceutically acceptable salt thereof, for the treatment of mast cell diseases and eosinophilic disorders.

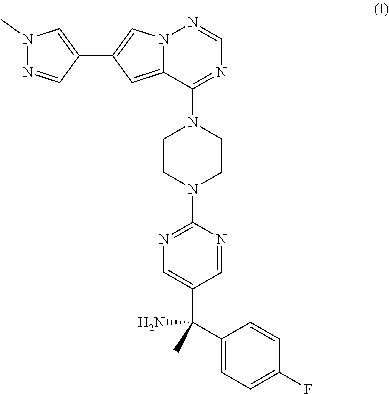 [SMILES: Cn1cc(-c2cc3c(N4CCN(c5ncc([C@@](C)(N)c6ccc(F)cc6)cn5)CC4)ncnn3c2)cn1]

### CROSS-REFERENCE TO RELATED APPLICATIONS

This application claims priority to U.S. Provisional Application No. 62/930,338, filed on Nov. 4, 2019. The entire contents of the aforementioned application are incorporated herein by reference.

### SUMMARY

It now has been found that Compound (I) as shown below is useful for the treatment of mast cell diseases and eosinophilic disorders.

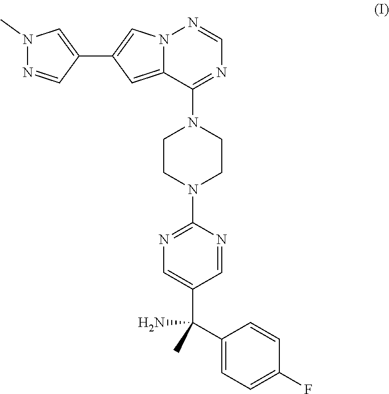 [SMILES: Cn1cc(-c2cc3c(N4CCN(c5ncc([C@@](C)(N)c6ccc(F)cc6)cn5)CC4)ncnn3c2)cn1]

One aspect of the present disclosure is a method of treating mast cell diseases, comprising administering to a subject in need thereof a therapeutically effective amount of Compound (I) or a pharmaceutically acceptable salt thereof.

Another aspect of the present disclosure is the use of Compound (I) or a pharmaceutically acceptable salt thereof for the manufacture of a medicament for treating mast cell diseases.

Another aspect of the present disclosure is Compound (I) or a pharmaceutically acceptable salt thereof for treating mast cell diseases.

One aspect of the present disclosure is a method of treating eosinophilic disorders, comprising administering to a subject in need thereof a therapeutically effective amount of Compound (I) or a pharmaceutically acceptable salt thereof.

Another aspect of the present disclosure is the use of Compound (I) or a pharmaceutically acceptable salt thereof for the manufacture of a medicament for treating eosinophilic disorders.

Another aspect of the present disclosure is Compound (I) or a pharmaceutically acceptable salt thereof for treating eosinophilic disorders.

### DETAILED DESCRIPTION

Compound (I) or a pharmaceutically acceptable salt thereof can be useful for treating mast cell diseases, including mast cell activation syndrome (MCAS), and hereditary alpha tryptasemia (HAT). Compound (I) can be useful for treating mast cell diseases associated with KIT and PDGFRa mutations. Compound (I) can be useful for treating mast cell diseases associated with wild type (WT) KIT.

Compound (I) or a pharmaceutically acceptable salt thereof can be useful for treating mast cell activation syndrome (MCAS), which is an immunological condition in which mast cells inappropriately and excessively release chemical mediators, resulting in a range of chronic symptoms, sometimes including anaphylaxis or near-anaphylaxis attacks. Unlike mastocytosis, where patients have an abnormally increased number of mast cells, patients with MCAS have a normal number of mast cells that do not function properly and are defined as “hyperresponsive.” Types of MCAS include primary MCAS (monoclonal mast cell activation syndrome (MMAS)), secondary MCAS (MCAS that arises from another disease), and idiopathic MCAS (MCAS that rules out primary or secondary MCAS).

Compound (I) or a pharmaceutically acceptable salt thereof can be useful for treating hereditary alpha tryptasemia (HAT)(overexpression of TPSAB1 causing elevated tryptase)).

Mast cells are involved in a variety of other diseases, such as urticaria (Maurer, Immunological Reviews (2018) 282: 232-247), anaphylaxis (Akin, C., Current Allergy and Asthma Review (2019) 19:31, and mast cell activation syndrome (Butterfield, J. Clin Immunol Pract (2019), 7 (4) 1097. Compound (I) or a pharmaceutically acceptable salt thereof can be useful for treating other mast cell diseases including mast cell mediated asthma, anaphylaxis (including idiopathic, Ig-E and non-Ig-E mediated), urticaria (including idiopathic and chronic), atopic dermatitis, swelling (angioedema), irritable bowel syndrome, mastocytic gastroenteritis, mastocytic colitis, pruritus, chronic pruritis, pruritis secondary to chronic kidney failure and heart, vascular, intestinal, brain, kidney, liver, pancreas, muscle, bone and skin conditions associated with mast cells.

Compounds (I) or a pharmaceutically acceptable salt thereof can also be useful for treating an eosinophilic disorders, including eosinophilic esophagitis, eosinophilic gastroenteritis, eosinophilic fasciitis, and Churg-Strauss syndrome.

The disclosure provides a method of treating a mast cell disease, comprising administering to a subject in need thereof an amount of 30 mg to 400 mg (e.g., 100 mg to 300 mg, or 200 mg to 300 mg) of Compound (I) and/or a pharmaceutically acceptable salt thereof once a day. In some embodiments, the amount is 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg once a day. In some embodiments, the amount is 25 mg once a day. In some embodiments, the amount is 50 mg once a day. In some embodiments, the amount is 75 mg once a day. In some embodiments, the amount is 100 mg once a day. In some embodiments, the amount is 150 mg once a day. In some embodiments, the amount is 200 mg once a day. In some embodiments, the amount is 250 mg once a day. In some embodiments, the amount is 300 mg once a day.

The disclosure provides a method of treating an eosinophilic disorder, comprising administering to a subject in need thereof an amount of 30 mg to 400 mg (e.g., 100 mg to 300 mg, or 200 mg to 300 mg) of Compound (I) and/or a pharmaceutically acceptable salt thereof once a day. In some embodiments, the amount is 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg once a day. In some embodiments, the amount is 25 mg once a day. In some embodiments, the amount is 50 mg once a day. In some embodiments, the amount is 75 mg once a day. In some embodiments, the amount is 100 mg once a day. In some embodiments, the amount is 150 mg once a day. In some embodiments, the amount is 200 mg once a day. In some embodiments, the amount is 250 mg once a day. In some embodiments, the amount is 300 mg once a day.

As used herein, “Compound (I)” refers to a compound having a chemical name (5)-1-(4-fluorophenyl)-1-(2-(4-(6-(1-methyl-1H-pyrazol-4-yl)pyrrolo[2,1-f][1,2,4]triazin-4-yl)piperazin-yl)pyrimidin-5-yl)ethan-1-amine, which has the following structure:

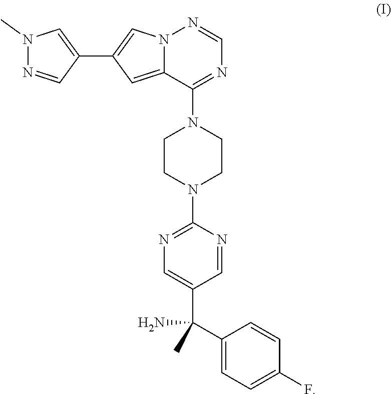 [SMILES: Cn1cc(-c2cc3c(N4CCN(c5ncc([C@@](C)(N)c6ccc(F)cc6)cn5)CC4)ncnn3c2)cn1]

Compound (I) is disclosed in WO 2015/057873, the entire teachings of which are incorporated herein by reference. The preparation of Compound (I) is described in Example 7 of WO 2015/057873.

Compound (I) was developed to selectively target KIT D816V and other KIT exon 17 mutations, and has demonstrated potent and selective activity against KIT D816V in vitro, robust growth inhibition in a tyrosine kinase inhibitor (TKI)-resistant mastocytoma model in vivo, and tolerability at active doses in toxicology and safety pharmacology studies. An ongoing Phase 1 study of Compound (I) in patients with advanced systemic mastocytosis (AdvSM) (NCT02561988) is evaluating safety and preliminary efficacy. The recommended Phase 2 dose (RP2D) was identified as 300 mg once a day (QD), and an expansion cohort of the study is further evaluating efficacy and safety of this dose in a larger cohort of patients, as well as validating the AdvSM Symptom Assessment Form (AdvSM-SAF) that has been developed to assess the impact of Compound (I) on symptom improvement in patients with AdvSM. Based on emerging safety and efficacy data in patients treated at 300 mg QD, an additional cohort of patients treated at 200 mg QD was added.

Activating mutations at the D816 position are found in eosinophilic disorders, with the most common mutations being D816V and D816Y. The D816V mutation is found in the activation loop of the kinase domain and leads to constitutive activation of KIT kinase.

Primary treatment with a KIT inhibitor such as imatinib has also been shown to be beneficial for initial treatment of eosinophilic disorders. Specifically, imatinib is approved for the treatment of idiopathic hypereosinophilic syndrome. However, resistance to imatinib occurs within months through somatic mutation. These secondary imatinib resistant mutations are most frequently located on Exon 11, 13, 14, 17 or 18. There is a need for therapeutic agents to treat patients with eosinophilic disorders, specifically patients who have exon 17 mutations.

Compound (I) or a pharmaceutically acceptable salt thereof can be active against one or more KIT mutations in Exon 17 (e.g., D816V, D816Y, D816F, D816K, D816H, D816A, D816G, D820A, D820E, D820G, N822K, N822H, Y823D, and A829P), and much less active against wild-type KIT.

In one embodiment, Compound (I) or a pharmaceutically acceptable salt thereof can be active against a D816 mutation in KIT in Exon 17. In a specific embodiment, the D816 mutation is D816V. In another specific embodiment, the D816 mutation is D816Y. Compound (I) shows greater potency against all disease-relevant KIT mutants than against WT KIT. Specifically, Compound (I) has been reported to have an IC50 value of 73 nM against WT KIT and an IC50 of 0.27 nM against D816V Exon 17 KIT (Evans et. al., Sci. Transl. Med. 9, eaao 1690 (2017). It is now known that Compound (I) can be useful for treating mast cell diseases associated with WT KIT. A mast cell disease associated with WT KIT means that a KIT mutation is not identified in a subject having the disease. Compound (I) can be useful for treating mast cell diseases in a subject who does not have a mutation in Exon 17 KIT. Compound (I) can be useful for treating mast cell diseases in a subject who does not have a D816V mutation in KIT. Compound (I) inhibits mast cell activation. In some embodiments, Compound (I) inhibits mast cell activation and degranulation. In some embodiments, the inhibition is in a dose-dependent manner. Compound (I) inhibits mast cell proliferation. Compound (I) does not interfere with the survival of mast cells i.e., Compound (I) does not kill mast cell. In some embodiments, Compound (I) both decreases mast cell burden and mast cell activation. Current treatment paradigms for mast cell diseases focus on inhibiting individual mast cell mediators for symptom control (anti-histamines, anti-IgE antibodies, etc.), or on using KIT exon 17 inhibitors in KIT mutant mMCAS.

As used herein, the term “pharmaceutically acceptable salt” refers to a non-toxic salt form of a compound of this disclosure. Pharmaceutically acceptable salts of Compound (I) include those derived from suitable inorganic and organic acids and bases. Pharmaceutically acceptable salts are well known in the art. Suitable pharmaceutically acceptable salts are, e.g., those disclosed in Berge, S. M., et al. J. Pharma. Sci. 66:1-19 (1977). Non-limiting examples of pharmaceutically acceptable salts disclosed in that article include: acetate; benzenesulfonate; benzoate; bicarbonate; bitartrate; bromide; calcium edetate; camsylate; carbonate; chloride; citrate; dihydrochloride; edetate; edisylate; estolate; esylate; fumarate; gluceptate; gluconate; glutamate; glycollylarsanilate; hexylresorcinate; hydrabamine; hydrobromide; hydrochloride; hydroxynaphthoate; iodide; isethionate; lactate; lactobionate; malate; maleate; mandelate; mesylate; methylbromide; methylnitrate; methyl sulfate; mucate; napsylate; nitrate; pamoate (embonate); pantothenate; phosphate/diphosphate; polygalacturonate; salicylate; stearate; subacetate; succinate; sulfate; tannate; tartrate; teociate; triethiodide; benzathine; chloroprocaine; choline; diethanolamine; ethylenediamine; meglumine; procaine; aluminum; calcium; lithium; magnesium; potassium; sodium; and zinc.

Non-limiting examples of pharmaceutically acceptable salts derived from appropriate acids include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid, or malonic acid; and salts formed by using other methods used in the art, such as ion exchange. Additional non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Non-limiting examples of pharmaceutically acceptable salts derived from appropriate bases include alkali metal, alkaline earth metal, ammonium, and N+(C1-4 alkyl)4 salts. This disclosure also envisions the quaternization of any basic nitrogen-containing groups of the compounds disclosed herein. Non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate, and aryl sulfonate. Other non-limiting examples of pharmaceutically acceptable salts include besylate and glucosamine salts.

As used herein, “a therapeutically effective amount” of a compound disclosed herein refers to an amount of the compound that will elicit a biological or medical response in a subject, e.g., reduce or inhibit enzyme or protein activity, ameliorate symptoms, alleviate conditions, or slow or delay disease progression.

As used herein, the term “patient” or “subject” refers to an organism to be treated by the methods of the disclosure. Non-limiting example organisms include mammals, e.g., murines, simians, equines, bovines, porcines, canines, felines, and the like. In some embodiments, the organism is a human.

As used herein, the term “treat,” “treating,” or “treatment,” when used in connection with a disorder or condition, includes any effect, e.g., lessening, reducing, modulating, ameliorating, and/or eliminating, that results in the improvement of the disorder or condition. Improvements in or lessening the severity of any symptom of the disorder or condition can be readily assessed according to standard methods and techniques known in the art.

### Pharmaceutical Compositions

Compound (I) and/or pharmaceutically acceptable salts thereof described herein are useful as an active pharmaceutical ingredients (API) as well as materials for preparing pharmaceutical compositions that incorporate one or more pharmaceutically acceptable excipients and is suitable for administration to human subjects.

In some embodiments, the disclosure provides a pharmaceutical composition comprising Compound (I) and/or a pharmaceutically acceptable salt thereof and at least one additional pharmaceutically acceptable excipient. The term “pharmaceutically acceptable excipient,” as used herein, refers to a pharmaceutically acceptable material, composition, and/or vehicle, such as a liquid or solid filler, diluent, excipient, solvent, or encapsulating material. Each excipient must be “pharmaceutically acceptable” in the sense of being compatible with the subject composition and its components and not injurious to the patient. Except insofar as any conventional pharmaceutically acceptable excipient is incompatible with Compound (I) and/or pharmaceutically acceptable salts thereof, such as by producing any undesirable biological effect or otherwise interacting in a deleterious manner with any other component(s) of the pharmaceutically acceptable composition, its use is contemplated to be within the scope of this disclosure.

Some non-limiting examples of materials which may serve as pharmaceutically acceptable excipients include: (1) sugars, such as lactose, glucose, and sucrose; (2) starches, such as corn starch and potato starch; (3) cellulose and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York, the contents of each of which is incorporated by reference herein, also disclose additional non-limiting examples of pharmaceutically acceptable excipients, as well as known techniques for preparing and using the same.

Pharmaceutical compositions disclosed herein may be administered orally, parenterally, by inhalation spray, topically, rectally, nasally, buccally, vaginally, or via an implanted reservoir. The term “parenteral,” as used herein includes subcutaneous, intravenous, intramuscular, intra-articular, intra-synovial, intrasternal, intrathecal, intrahepatic, intralesional, and intracranial injection or infusion techniques. In some embodiments, the compositions of the disclosure are administered orally, intraperitoneally, or intravenously. Sterile injectable forms of the pharmaceutical compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example, as a solution in 1,3-butanediol. Non-limiting examples of acceptable vehicles and solvents that may be employed are water, Ringer's solution, and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives, are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tween, Spans, and other emulsifying agents or bioavailability enhancers which are commonly used in the manufacture of pharmaceutically acceptable solid, liquid, or other dosage forms may also be used for the purposes of formulation.

Pharmaceutical compositions disclosed herein may also be orally administered in any orally acceptable dosage form including, but not limited to, capsules, tablets, aqueous suspensions, or solutions. When aqueous suspensions are required for oral use, the active ingredient is typically combined with emulsifying and suspending agents. If desired, certain sweetening, flavoring, or coloring agents may also be added. In some embodiments, the pharmaceutical composition comprising Compound (I) and/or a pharmaceutically acceptable salt thereof is a tablet prepared using methods known in the art. In some embodiments, the tablet is an immediate release tablet for oral administration. In some embodiments, Compound (I) and/or a pharmaceutically acceptable salt thereof is blended with pharmacopeial excipients to form an immediate release tablet. In some embodiments, the excipients comprising the tablet are microcrystalline cellulose, copovidone, croscarmellose sodium, and magnesium sterate. In some embodiments, the formulation blend is roller compacted, compressed into round tablets, and aesthetically film coated.

1. A method of treating a mast cell disease, comprising administering to a subject in need thereof a therapeutically effective amount of Compound (I): 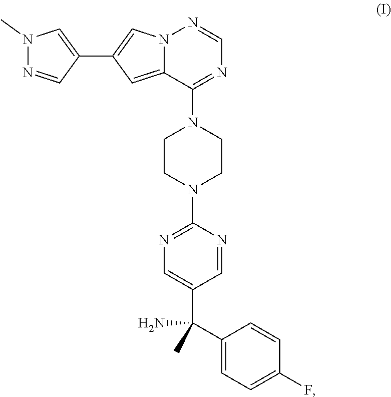 [SMILES: Cn1cc(-c2cc3c(N4CCN(c5ncc([C@@](C)(N)c6ccc(F)cc6)cn5)CC4)ncnn3c2)cn1]
or a pharmaceutically acceptable salt thereof.

2. The method of claim 1, wherein the mast cell disease is selected from mast cell activation syndrome (MCAS), and hereditary alpha tryptasemia (HAT).

3. The method of claim 2, wherein the MCAS is selected from primary MCAS (monoclonal mast cell activation syndrome (MMAS)), secondary MCAS (MCAS that arises from another disease), and idiopathic MCAS (MCAS that rules out primary or secondary MCAS).

4. The method of claim 2, wherein the mast cell disease is hereditary alpha tryptasemia (HAT)(overexpression of TPSAB1 causing elevated tryptase)).

5. The method of claim 2, wherein the mast cell disease is selected from mast cell mediated asthma, anaphylaxis (including idiopathic, Ig-E and non-Ig-E mediated), urticaria (including idiopathic and chronic), atopic dermatitis, swelling (angioedema), irritable bowel syndrome, mastocytic gastroenteritis, mastocytic colitis, pruritus, chronic pruritis, pruritis secondary to chronic kidney failure and heart, vascular, intestinal, brain, kidney, liver, pancreas, muscle, bone and skin conditions associated with mast cells.

6. The method of claim 1, wherein the mast cell disease is associated with wild type KIT.

7. The method of claim 1, wherein the subject does not have a mutation in Exon 17 KIT.

8. The method of claim 7, wherein the subject does not have a D816V mutation in KIT.

9. A method of treating an eosinophilic disorder, comprising administering to a subject in need thereof a therapeutically effective amount of Compound (I): 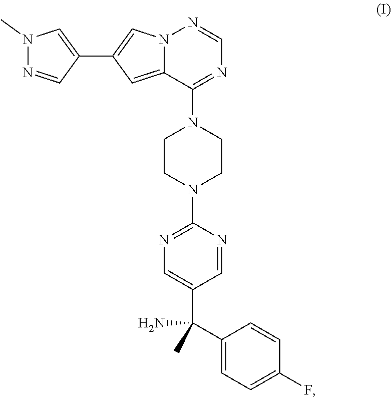 [SMILES: Cn1cc(-c2cc3c(N4CCN(c5ncc([C@@](C)(N)c6ccc(F)cc6)cn5)CC4)ncnn3c2)cn1]
or a pharmaceutically acceptable salt thereof.

10. The method of claim 9, wherein the eosinophilic disorders is selected from eosinophilic esophagitis, eosinophilic gastroenteritis, eosinophilic fasciitis, and Churg-Strauss syndrome.

11. The method of claim 1, wherein the subject has a mutation in Exon 17 in KIT.

12. The method of claim 11, wherein the subject has a D816 mutation in KIT in Exon 17.

13. The method of claim 12, wherein the D816 mutation is D816V.

14. The method of claim 12, wherein the D816 mutation is D816Y.

15. The method of claim 1, wherein the therapeutically effective amount is 30-400 mg.

16. The method of claim 15, wherein the therapeutically effective amount is 100-300 mg per day.

17. The method of claim 16, wherein the therapeutically effective amount is 100 mg per day.

18. The method of claim 16, wherein the therapeutically effective amount is 200 mg per day.

19. The method of claim 16, wherein the therapeutically effective amount is 300 mg per day.
